SEQUENCE LISTING (1 sequence)

SEQ ID NO: 1 - protein, 192 aa, Homo sapiens
MQAIKCVVVGDGAVGKTCLLISYTTNAFPGEYIPTVFDNYSANVMVDSKPVNLGLWDTAGQEDYDRLRPLSYPQTDVFLICFSLVSPASYENVRAKWFPEVRHHCPSTPIILVGTKLDLRDDKDTIEKLKEKKLAPITYPQGLALAKEIDSVKYLECSALTQRGLKTVFDEAIRAVLCPQPTRQQKRACSLL

(19) United States
(12) Patent Application Publication
(10) Pub. No.: US 2025/0084389 A1
(43) Pub. Date: Mar. 13, 2025

(54) METHODS OF INDUCING CELL DEATH OF A POPULATION OF SOLID TUMOR CELLS

(71) Applicants: INSTITUT NATIONAL DE LA SANTÉ ET DE LA RECHERCHE MÉDICALE, Paris (FR); FONDATION IMAGINE, Paris (FR); ASSISTANCE PUBLIQUE-HÔPITAUX DE PARIS (APHP), Paris (FR); UNIVERSITÉ PARIS CITÉ, Paris (FR)

(72) Inventors: Chantal LAGRESLE-PEYROU, Paris (FR); Marina CAVAZZANA, Paris (FR); Isabelle ANDRE, Paris (FR); Boris BESSOT, Paris (FR); Nataël SOREL, Paris (FR)

(21) Appl. No.: 18/725,322

(22) PCT Filed: Jan. 16, 2023
(86) PCT No.: PCT/EP2023/050871

§ 371 Date: Jun. 28, 2024

(30) Foreign Application Priority Data
Jan. 17, 2022 (EP) 22305037.8

(51) Int. Cl. C12N 9/14; A61K 38/00; C12N 15/86

(52) U.S. Cl. CPC C12N 9/14; C12N 15/86; A61K 38/00; C12N 2740/15043; C12Y 306/05002

(57) ABSTRACT

It was previously demonstrated that the RAC2 G12R mutation rapidly induced HSPCs cell death and hematopoiesis regulation. Now, the invention evaluated the impact of said mutation on three tumor cell lines: MDA-MB-231 (mammary gland adeno-carcinoma), HT29 (colorectal adenocarcinoma) and HepG2 (hepatocellular carcinoma). Briefly, cells were transduced with a lentiviral vector containing the green fluorescent protein (GFP) reporter cDNA (WPI) or a wild type form of RAC2 cDNA (WT) or a RAC2 mutated cDNA form (G12R). The inventors showed that the number of GFP+ cells is drastically lower in the G12R condition as compared to the WT and WPI conditions. The cell morphology and content are particularly disrupted. These observations were confirmed in a time-course proliferation assay performed on MDA-MB-231 and HT29 cell lines. Altogether, these data underlie the deleterious impact of the RAC2 G12R mutation on tumor cell lines proliferation.

### FIELD OF THE INVENTION

The present invention is in the field of medicine, in particular oncology.

### BACKGROUND OF THE INVENTION

Cancer is one of the leading causes of death. For instance, in 2019, there were 599,601 cancer deaths; 283,725 were among females and 315,876 among males (Centers for Disease Control and Prevention. An Update on Cancer Deaths in the United States. Atlanta, GA: US Department of Health and Human Services, Centers for Disease Control and Prevention, Division of Cancer Prevention and Control; 2021). Lung cancer was the leading cause of cancer death, accounting for 23% of all cancer deaths in USA in 2019. Other common causes of cancer death were cancers of the colon and rectum (9%), pancreas (8%), female breast (7%), prostate (5%), and liver and intrahepatic bile duct (5%). Other cancers individually accounted for less than 5% of cancer deaths. There is thus a need for new therapies of cancer.

Recently, an autosomal dominant (AD) missense mutation (i.e. the p.G12R) was identified in the RAC2 gene (coding for Ras-related C3 botulinum toxin substrate 2 (RAC2)) in three Severe combined immunodeficiencies (SCID) patients whose clinical presentation overlaps with the RD SCID form but who lack AK2 mutations and deafness (Lagresle-Peyrou C, Olichon A, Sadek H, Roche P, Tardy C, Da Silva C, Garrigue A, Fischer A, Moshous D, Collette Y, Picard C, Casanova J L, André I, Cavazzana M. A gain-of-function RAC2 mutation is associated with bone-marrow hypoplasia and an autosomal dominant form of severe combined immunodeficiency. Haematologica. 2021 Feb. 1; 106(2):404-411). The RAC2 mutation was closely related to an impairment in cell differentiation capacity and defects in cellular and mitochondrial networks. Therefore, WO 2021/009336 teaches a method of inducing cell death of a population of malignant hematopoietic cells comprising contacting said population with an effective amount of i) a RAC2 polypeptide comprising the p.G12R mutation, or ii) a polynucleotide encoding for such a polypeptide.

### SUMMARY OF THE INVENTION

The present invention is defined by the claims. In particular, the present invention relates to methods of inducing cell death of a population of solid tumor cells.

### DETAILED DESCRIPTION OF THE INVENTION

The first object of the present invention relates to a method of inducing cell death of a population of solid tumor cells comprising contacting said population with an effective amount of i) a polypeptide comprising the amino acid sequence as set forth in SEQ ID NO: 1 wherein the amino residue (G) at position 12 is mutated, or ii) a polynucleotide encoding for a polypeptide comprising the amino acid sequence as set forth in SEQ ID NO:1 wherein the amino residue (G) at position 12 is mutated.

A further object of the present invention relates to a method of treating a solid tumor in a patient in need thereof comprising administering to the patient a therapeutically effective amount of i) a polypeptide comprising the amino acid sequence as set forth in SEQ ID NO:1 wherein the amino residue (G) at position 12 is mutated, or ii) a polynucleotide encoding for a polypeptide comprising the amino acid sequence as set forth in SEQ ID NO:1 wherein the amino residue (G) at position 12 is mutated.

As used herein, the term “solid cancer” has its general meaning in the art and refers to one or more cells which are growing or have grown in an uncontrolled manner to form cancer tissue. As used herein, the term “solid cancer” includes, but is not limited to “carcinomas”, “adenocarcinomas” and “sarcomas”. “Sarcomas” are cancers of the connective tissue, cartilage, bone, muscle, and so on. “Carcinomas” are cancers of epithelial (lining) cells. “Adenocarcinoma” refers to carcinoma derived from cells of glandular origin. The terms “cancer” and “tumor” are used interchangeably throughout the subject specification. The term “cancer” is not limited to any stage, grade, histomorphological feature, invasiveness, aggressiveness or malignancy of an affected tissue or cell aggregation. In particular stage 0 cancer, stage I cancer, stage II cancer, stage III cancer, stage IV cancer, grade I cancer, grade II cancer, grade III cancer, malignant cancer and primary carcinomas are included.

Typically the patient subjected to the above method may suffer from a solid cancer selected from the group consisting adrenal cortical cancer, anal cancer, bile duct cancer (e.g. periphilar cancer, distal bile duct cancer, intrahepatic bile duct cancer), bladder cancer, bone cancer (e.g. osteoblastoma, osteochrondroma, hemangioma, chondromyxoid fibroma, osteosarcoma, chondrosarcoma, fibrosarcoma, malignant fibrous histiocytoma, giant cell tumor of the bone, chordoma, multiple myeloma), brain and central nervous system cancer (e.g. meningioma, astocytoma, oligodendrogliomas, ependymoma, gliomas, medulloblastoma, ganglioglioma, Schwannoma, germinoma, craniopharyngioma), breast cancer (e.g. ductal carcinoma in situ, infiltrating ductal carcinoma, infiltrating lobular carcinoma, lobular carcinoma in situ, gynecomastia), cervical cancer, colorectal cancer, endometrial cancer (e.g. endometrial adenocarcinoma, adenocanthoma, papillary serous adnocarcinoma, clear cell), esophagus cancer, gallbladder cancer (mucinous adenocarcinoma, small cell carcinoma), gastrointestinal carcinoid tumors (e.g. choriocarcinoma, chorioadenoma destruens), Kaposi's sarcoma, kidney cancer (e.g. renal cell cancer), laryngeal and hypopharyngeal cancer, liver cancer (e.g. hemangioma, hepatic adenoma, focal nodular hyperplasia, hepatocellular carcinoma), lung cancer (e.g. small cell lung cancer, non-small cell lung cancer), mesothelioma, plasmacytoma, nasal cavity and paranasal sinus cancer (e.g. esthesioneuroblastoma, midline granuloma), nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma (e.g. embryonal rhabdomyosarcoma, alveolar rhabdomyosarcoma, pleomorphic rhabdomyosarcoma), salivary gland cancer, skin cancer (e.g. melanoma, nonmelanoma skin cancer), stomach cancer, testicular cancer (e.g. seminoma, nonseminoma germ cell cancer), thymus cancer, thyroid cancer (e.g. follicular carcinoma, anaplastic carcinoma, poorly differentiated carcinoma, medullary thyroid carcinoma), vaginal cancer, vulvar cancer, and uterine cancer (e.g. uterine leiomyosarcoma).

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein, the term “Rac2” has its general meaning in the art and refers to Ras-related C3 botulinum toxin substrate 2. An exemplary amino acid sequence for the human Rac2 is represented by SEQ ID NO:1.

>sp|P15153|Rac2_HUMAN Ras-related C3 botulinum toxin substrate
2 OS = Homo sapiens OX = 9606 GN = Rac2 PE = 1 SV = 1
SEQ ID NO: 1
MQAIKCVVVGDGAVGKTCLLISYTTNAFPGEYIPTVFDNYSANVMVDSKPVNLGLWDTAG
QEDYDRLRPLSYPQTDVELICFSLVSPASYENVRAKWFPEVRHHCPSTPIILVGTKLDLR
DDKDTIEKLKEKKLAPITYPQGLALAKEIDSVKYLECSALTQRGLKTVFDEAIRAVLCPQ
PTRQQKRACSLL

As used herein, the term “mutation” has its general meaning in the art and refers to a substitution, deletion or insertion. The term “substitution” means that a specific amino acid residue at a specific position is removed and another amino acid residue is inserted into the same position. The term “deletion” means that a specific amino acid residue is removed. The term “insertion” means that one or more amino acid residues are inserted before or after a specific amino acid residue, more specifically, that one or more, preferably one or several, amino acid residues are bound to an a.-carboxyl group or an a,-amino group of the specific amino acid residue.

In some embodiments, the amino residue (G) at position 12 is substituted. In some embodiments, the amino residue (G) at position 12 is substituted by an amino acid residue (R). As used herein, the term “polypeptide” has its general meaning in the art and refers to a polymer of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

As used herein, the term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In some embodiments, the polynucleotide comprises an mRNA. In other aspect, the mRNA is a synthetic mRNA. In some embodiments, the synthetic mRNA comprises at least one unnatural nucleobase. In some embodiments, all nucleobases of a certain class have been replaced with unnatural nucleobases (e.g., all uridines in a polynucleotide disclosed herein can be replaced with an unnatural nucleobase, e.g., 5-methoxyuridine). In some embodiments, the polynucleotide (e.g., a synthetic RNA or a synthetic DNA) comprises only natural nucleobases, i.e., A, C, T and G in the case of a synthetic DNA, or A, C, T, and U in the case of a synthetic RNA.

In some embodiments, the polynucleotide of the present invention is a messenger RNA (mRNA).

In some embodiments, the polynucleotide is inserted in a vector, such as a plasmid, cosmid, episome, artificial chromosome, phage or a viral vector. Typically, the vector is a viral vector which is an adeno-associated virus (AAV), a retrovirus, bovine papilloma virus, an adenovirus vector, a lentiviral vector, a vaccinia virus, a polyoma virus, or an infective virus. Typically, the vector of the present invention include “control sequences”, which refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell. Another nucleic acid sequence, is a “promoter” sequence, which is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters”.

In some embodiments, the polypeptide or polynucleotide of the present invention can be conjugated to at least one other molecule. Typically, said molecule is selected from the group consisting of polynucleotides, polypeptides, lipids, lectins, carbohydrates, vitamins, cofactors, and drugs.

By a “therapeutically effective amount” is meant a sufficient amount of the active ingredient for treating or reducing the symptoms at reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination with the active ingredients; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically the active ingredient of the present invention (i.e. the polypeptide or polynucleotide) is combined with pharmaceutically acceptable excipients, and optionally sustained-release matrices, such as biodegradable polymers, to form pharmaceutical compositions. The term “Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In some embodiments, the polypeptide or polynucleotide of the present invention is formulated with lipidoids. The synthesis of lipidoids has been extensively described (see Mahon et al., Bioconjug Chem. 2010 21:1448-1454; Schroeder et al., J Intern Med. 2010 267:9-21; Akinc et al., Nat Biotechnol. 2008 26:561-569; Love et al., Proc Natl Acad Sci USA. 2010 107:1864-1869; Siegwart et al., Proc Natl Acad Sci US A. 2011 108:12996-3001). While these lipidoids have been used to effectively deliver double stranded small interfering RNA molecules in rodents and non-human primates (see Akinc et al., Nat Biotechnol. 2008 26:561-569; Frank-Kamenetsky et al., Proc Natl Acad Sci USA. 2008 105:11915-11920; Akinc et al., Mol Ther. 2009 17:872-879; Love et al., Proc Natl Acad Sci USA. 2010 107:1864-1869; Leuschner et al., Nat Biotechnol. 2011 29:1005-1010), the present disclosure describes their formulation and use in delivering polynucleotides.

In some embodiments, the polypeptide or polynucleotide of the present invention is formulated using one or more lipid-based structures that include but are not limited to liposomes, lipoplexes, or lipid nanoparticles (Paunovska, Kalina, David Loughrey, and James E. Dahlman. “Drug delivery systems for RNA therapeutics.” Nature Reviews Genetics (2022): 1-16).

Liposomes are artificially-prepared vesicles which can primarily be composed of a lipid bilayer and can be used as a delivery vehicle for the administration of pharmaceutical formulations. Liposomes can be of different sizes such as, but not limited to, a multilamellar vesicle (MLV) which can be hundreds of nanometers in diameter and can contain a series of concentric bilayers separated by narrow aqueous compartments, a small unicellular vesicle (SUV) which can be smaller than 50 nm in diameter, and a large unilamellar vesicle (LUV) which can be between 50 and 500 nm in diameter. Liposome design can include, but is not limited to, opsonins or ligands in order to improve the attachment of liposomes to unhealthy tissue or to activate events such as, but not limited to, endocytosis. Liposomes can contain a low or a high pH in order to improve the delivery of the pharmaceutical formulations. As a non-limiting example, liposomes such as synthetic membrane vesicles are prepared by the methods, apparatus and devices described in US Patent Publication No. US20130177638, US20130177637, US20130177636, US20130177635, US20130177634, US20130177633, US20130183375, US20130183373 and US20130183372. In some embodiments, the liposomes are formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (as described in US20100324120) and liposomes which can deliver small molecule drugs such as, but not limited to, DOXIL® from Janssen Biotech, Inc. (Horsham, Pa.). The polypeptide of polynucleotide of the present invention can be encapsulated by the liposome and/or it can be contained in an aqueous core which can then be encapsulated by the liposome (see International Pub. Nos. WO2012031046, WO2012031043, WO2012030901 and WO2012006378 and US Patent Publication No. US20130189351, US20130195969 and US20130202684).

In some embodiments, the polynucleotide of the present invention is formulated with stabilized plasmid-lipid particles (SPLP) or stabilized nucleic acid lipid particle (SNALP) that have been previously described and shown to be suitable for oligonucleotide delivery in vitro and in vivo (see Wheeler et al. Gene Therapy. 1999 6:271-281; Zhang et al. Gene Therapy. 1999 6:1438-1447; Jeffs et al. Pharm Res. 2005 22:362-372; Morrissey et al., Nat Biotechnol. 2005 2:1002-1007; Zimmermann et al., Nature. 2006 441:111-114; Heyes et al. J Contr Rel. 2005 107:276-287; Semple et al. Nature Biotech. 2010 28:172-176; Judge et al. J Clin Invest. 2009 119:661-673; deFougerolles Hum Gene Ther. 2008 19:125-132; U.S. Patent Publication No US20130122104). The original manufacture method by Wheeler et al. was a detergent dialysis method, which was later improved by Jeffs et al. and is referred to as the spontaneous vesicle formation method. The liposome formulations are composed of 3 to 4 lipid components in addition to the polynucleotide. As an example a liposome can contain, but is not limited to, 55% cholesterol, 20% disteroylphosphatidyl choline (DSPC), 10% PEG-S-DSG, and 15% 1,2-dioleyloxy-N,N-dimethylaminopropane (DODMA), as described by Jeffs et al. As another example, certain liposome formulations contain, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA), as described by Heyes et al.

In some embodiments, the polynucleotide of the present invention is formulated in a lipid nanoparticle such as those described in International Publication No. WO2012170930. Lipid nanoparticle formulations typically comprise a lipid, in particular, an ionizable cationic lipid, and further comprise a neutral lipid, a sterol and a molecule capable of reducing particle aggregation, for example a PEG or PEG-modified lipid. The lipid can be selected from, but is not limited to, DLin-DMA, DLin-K-DMA, 98N12-5, C12-200, DLin-MC3-DMA, DLin-KC2-DMA, DODMA, PLGA, PEG, PEG-DMG, PEGylated lipids and amino alcohol lipids. In some embodiments, the lipid is a cationic lipid such as, but not limited to, DLin-DMA, DLin-D-DMA, DLin-MC3-DMA, DLin-KC2-DMA, DODMA and amino alcohol lipids. The amino alcohol cationic lipid can be the lipids described in and/or made by the methods described in US Patent Publication No. US20130150625. As a non-limiting example, the cationic lipid can be 2-amino-3-[(9Z,12Z)-octadeca-9,12-dien-1-yloxy]-2-{[(9Z,2Z)-octadeca-9,12-dien-1-yloxy]methyl}propan-1-ol (Compound 1 in US20130150625); 2-amino-3-[(9Z)-octadec-9-en-1-yloxy]-2-{[(9Z)-octadec-9-en-1-yloxy]methyl}propan-1-ol (Compound 2 in US20130150625); 2-amino-3-[(9Z,12Z)-octadeca-9,12-dien-1-yloxy]-2-[(octyloxy)methyl]propan-1-ol (Compound 3 in US20130150625); and 2-(dimethylamino)-3-[(9Z,12Z)-octadeca-9,12-dien-1-yloxy]-2-{[(9Z,12Z)-octadeca-9,12-dien-1-yloxy]methyl}propan-1-ol (Compound 4 in US20130150625); or any pharmaceutically acceptable salt or stereoisomer thereof. Nanoparticle formulations of the present disclosure can be coated with a surfactant or polymer in order to improve the delivery of the particle. In some embodiments, the nanoparticle is coated with a hydrophilic coating such as, but not limited to, PEG coatings and/or coatings that have a neutral surface charge. The hydrophilic coatings can help to deliver nanoparticles with larger payloads such as, but not limited to, polynucleotides within the central nervous system. As a non-limiting example nanoparticles comprising a hydrophilic coating and methods of making such nanoparticles are described in US Patent Publication No. US20130183244.

The invention will be further illustrated by the following figures and examples. However, these examples and figures should not be interpreted in any way as limiting the scope of the present invention.

### FIGURES

FIG. 1: RAC2 G12R mutation impaired solid tumor cell lines proliferation.

A) Evaluation of the proliferative capacity of three solid tumor cell lines (MDA-MB-231, HT29 and HepG2) after a 5 and 8-day in vitro culture (grey and black histograms, respectively). The alive GFP+ cell number was evaluated after lentiviral transduction with the fluorescent protein (GFP) reporter cDNA (WPI), the wild type form of RAC2 cDNA (WT) or the RAC2 mutated cDNA form (G12R). For each cell line, the histograms are representative of three independent experiments.

B) In vitro kinetic proliferation assay of MDA-MB-231 (left panel) and HT29 (right panel) cell lines either non-transduced (NT) or transduced with the WPI, WT or G12R lentiviral constructs.

The GFP+ cell growth (measured as the % of confluence with the green filter) was evaluated every 3 hours for 14 days. For each cell line, the curves are representative of three independent experiments.

### EXAMPLE: RAC2 G12R MUTATION IMPAIRED TUMOR SOLID CELL LINES PROLIFERATION

Severe Combined Immune Deficiencies (SCID) are inherited disorders characterized by a blockade in T lymphoid differentiation associated with an absence or a functional defect of B NK-cell or neutrophil lineages¹. In three newborns presenting with frequent infections and profound leukopenia, we identified a private, heterozygous mutation in the RAC2 gene (coding for Ras-related C3 botulinum toxin substrate 2). RAC2 protein belongs to the Rac subfamily of RHO small GTPases and is finely regulated. In the inactive GDP-bound state, RAC2 is located in the cytosol and upon stimulation, the active RAC2-GTP-bound form translocates to the plasma membrane². Unlike the other members of the Rac subfamily (RAC1 and RAC3), RAC2 is mostly expressed on hematopoietic cells^{3,4}.

The missense mutation we identified is located at position 12 of the protein (p.G12R), in the highly conserved guanine nucleotide binding region required for GTP hydrolysis and RAC2 signaling pathways. We demonstrated that the mutation has an impact on the catalytic domain of the protein and induced an abnormally high and sustained level of the GTP-binding RAC2 state. Moreover, this gain of function mutation rapidly blocks cord blood hematopoietic stem/progenitor cells (HSPCs) proliferation and differentiation toward the T lymphoid, neutrophil and monocyte lineages. Such observations are associated with defective mitochondria function, disturbed reactive oxygen species (ROS) production and high apoptosis. Taken as a whole, the RAC2 G12R mutation has a strong impact on the homeostatic regulation of hematopoiesis, which could explain the severity of the patients' clinical and immunological phenotypes⁵. This present study is the first to describe an autosomal SCID form and RAC2 gene sequencing is now included in newborn screening programs for SCID detection⁶ at the Necker Hospital (Paris, France). Of note, all the other RAC2 mutations previously described in the literature did not have such a drastic impact on HSPCs fate⁷.

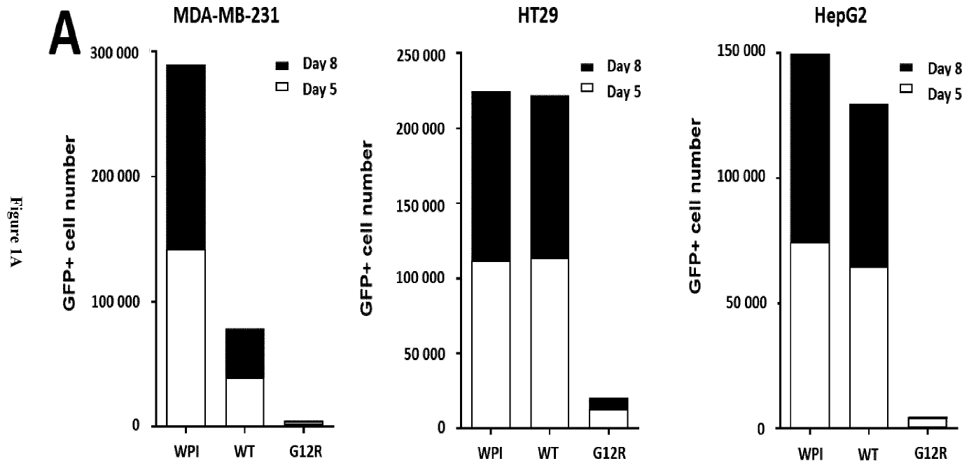

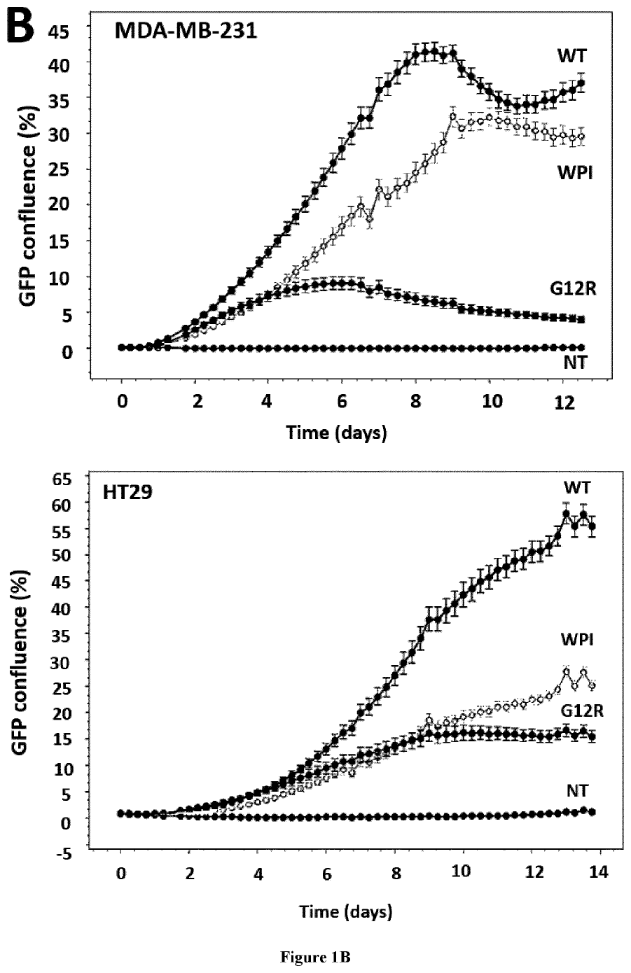

We described above that RAC2 G12R mutation rapidly induced HSPCs cell death and hematopoiesis regulation. Using the same lentiviral vector approach, we evaluated the impact of RAC2 G12R mutation on three tumor cell lines: MDA-MB-231 (mammary gland adenocarcinoma), HT29 (colorectal adenocarcinoma) and HepG2 (hepatocellular carcinoma). Briefly, cells were transduced with a lentiviral vector containing the green fluorescent protein (GFP) reporter cDNA (WPI) or a wild type form of RAC2 cDNA (WT) or a RAC2 mutated cDNA form (G12R). After the transduction step, the cells were cultured in complete medium for up to 8 days (FIG. 1A). At day 5 and day 8, the number of GFP positive (GFP+) cells was evaluated after total cell number count (trypan blue staining to remove dead cell) and immunofluorescence analysis. Whatever the day and the cell line, the number of GFP+ cells is drastically lower in the G12R condition as compared to the WT and WPI conditions. The cell morphology and content are particularly disrupted as observed by a live cell imaging microscope measuring the quantitative refractive index of the cells (data not shown). To confirm these observations, a time-course proliferation assay was performed on MDA-MB-231 and HT29 cell lines. The GFP+ transduced cell growth (expressed as the % of confluence observed with the green filter) was measured every 3 hours for 12 days using the Incucyte live-cell analysis system. For the MDA-MB-231 cell line, we observed an exponential GFP+ cell growth in the WPI and WT conditions and a flat curve in the G12R condition (FIG. 1B, up panel). For the HT29 cell line, the exponential GFP+ cell growth is higher in the WT condition as compared to the WPI and G12R conditions. Of note after day 8, the curve has reached a plateau (10% confluence) in the G12R condition, suggesting that most of the GFP transduced cells were dead. In the WT and WPI conditions, the % of confluence increased over time and after day 12 reached 65% and 25%, respectively (FIG. 1B, down panel).

Altogether, these data underlie the deleterious impact of the RAC2 G12R mutation on tumor cell lines proliferation.

### REFERENCES

Throughout this application, various references describe the state of the art to which this invention pertains. The disclosures of these references are hereby incorporated by reference into the present disclosure.
1-. Picard C, Al-Herz W, Bousfiha A, Casanova J L, Chatila T, Conley M E, Cunningham-Rundles C, Etzioni A, Holland S M, Klein C, Nonoyama S, Ochs H D, Oksenhendler E, Puck J M, Sullivan K E, Tang M L, Franco J L, Gaspar H B. Primary immunodeficiency diseases: an update on the classification from the International Union of Immunological Societies Expert Committee for Primary Immunodeficiency 2015. J Clin Immunol. 2015; 35(8):696-726. doi: 10.1007/s10875-015-0201-1
2. Hodge R G, Ridley A J. Regulating Rho GTPases and their regulators. Nat Rev Mol Cell Biol. août 2016; 17(8):496-510.
3. Gu Y, Filippi M-D, Cancelas J A, Siefring J E, Williams E P, Jasti A C, et al. Hematopoietic cell regulation by Rac1 and Rac2 guanosine triphosphatases. Science. 17 Oct. 2003; 302(5644):445-9.
4. Shirsat N V, Pignolo R J, Kreider B L, Rovera G. A member of the ras gene superfamily is expressed specifically in T, B and myeloid hemopoietic cells. Oncogene. mai 1990; 5(5):769-72.
5. Lagresle-Peyrou C, Olichon A, Sadek H, Roche P, Tardy C, Silva C D, et al. A gain-of-function RAC2 mutation is associated with bone-marrow hypoplasia and an autosomal dominant form of severe combined immunodeficiency. Haematologica. 2021; 106(2):404-11.
6. Fusaro M, Rosain J, Grandin V, Lambert N, Hanein S, Fourrage C, et al. Improving the diagnostic efficiency of primary immunodeficiencies with targeted next-generation sequencing. J Allergy Clin Immunol. févr 2021; 147(2):734-7.
7. Lougaris V, Baronio M, Gazzurelli L, Benvenuto A, Plebani A. RAC2 and primary human immune deficiencies. J Leukoc Biol. 2020; 108(2):687-96.

1. A method of inducing cell death of a population of solid tumor cells comprising contacting said population with an effective amount of i) a polypeptide comprising the amino acid sequence as set forth in SEQ ID NO:1 wherein the amino residue (G) at position 12 is mutated, or ii) a polynucleotide encoding for a polypeptide comprising the amino acid sequence as set forth in SEQ ID NO:1 wherein the amino residue (G) at position 12 is mutated.

2. A method of treating a solid tumor in a patient in need thereof comprising administering to the patient a therapeutically effective amount of i) a polypeptide comprising the amino acid sequence as set forth in SEQ ID NO:1 wherein the amino residue (G) at position 12 is mutated, or ii) a polynucleotide encoding for a polypeptide comprising the amino acid sequence as set forth in SEQ ID NO:1 wherein the amino residue (G) at position 12 is mutated.

3. The method according to any one of claim 1, wherein the amino residue (G) at position 12 is substituted.

4. The method of claim 3 wherein the amino residue (G) at position 12 is substituted by an amino acid residue (R).

5. The method according to claim 1, wherein the polynucleotide is a messenger RNA (mRNA).

6. The method according to claim 1, wherein the polynucleotide is inserted in a vector.

7. The method according to claim 1, wherein the polypeptide or the polynucleotide is conjugated to at least one other molecule selected from the group consisting of polynucleotides, polypeptides, lipids, lectins, carbohydrates, vitamins, cofactors, and drugs.

8. The method according to claim 1, wherein the polypeptide or the polynucleotide is formulated using one or more lipid-based structures that are liposomes, lipoplexes, or lipid nanoparticles.

9. The method according to claim 2, wherein the amino residue (G) at position 12 is substituted.

10. The method of claim 9, wherein the amino residue (G) at position 12 is substituted by an amino acid residue (R).

11. The method according to claim 2, wherein the polynucleotide is a messenger RNA (mRNA).

12. The method according to claim 2, wherein the polynucleotide is inserted in a vector.

13. The method according to claim 2, wherein the polypeptide or the polynucleotide is conjugated to at least one other molecule selected from the group consisting of polynucleotides, polypeptides, lipids, lectins, carbohydrates, vitamins, cofactors, and drugs.

14. The method according to claim 2, wherein the polypeptide or the polynucleotide is formulated using one or more lipid-based structures that are liposomes, lipoplexes, or lipid nanoparticles.
